(19) United States
(12) Patent Application Publication
(10) Pub. No.: US 2024/0307209 A1
(43) Pub. Date: Sep. 19, 2024

(54) PLATFORM DEVICE TO SUPPORT BREASTS

(71) Applicant: Tara Ramsey, Lawrenceville, GA (US)

(72) Inventor: Tara Ramsey, Lawrenceville, GA (US)

(21) Appl. No.: 18/121,585

(22) Filed: Mar. 14, 2023

(51) Int. Cl. A61F 5/03

(52) U.S. Cl. CPC A61F 5/03

(57) ABSTRACT

Implementations of a platform device to support breasts comprise a base, a vertical pole, and a platform. In some implementations, a method of using the platform device to support breasts comprises positioning the base of the platform device to support breasts on a user's lap, on a sitting surface under a user's thighs, or on another suitable surface, further positioning the platform device to support breasts adjacent to and/or against the user's body, and resting the user's breast on the platform of the platform device to support breasts.

### TECHNICAL FIELD

This disclosure relates to implementations of a platform device to support breasts to relieve pressure on a user's body caused by the user's breast.

### BACKGROUND

Problems caused by large breasts include chronic pain in the neck, back, and shoulders, such as from pressure on the upper body caused by the large breasts.

### BRIEF DESCRIPTION OF THE DRAWINGS

FIGS. 1-13 illustrate implementations of an example platform device to support breasts according to the present disclosure.

FIG. 14 illustrates another implementation of an example platform device to support breasts according to the present disclosure.

FIG. 15 illustrates another implementation of an example platform of the platform device to support breasts according to the present disclosure.

FIG. 16 illustrates an example use of the platform device to support breasts according to the present disclosure.

### DETAILED DESCRIPTION

Implementations of a platform device to support breasts are provided. In some implementations, the platform device to support breasts comprises a base, a vertical pole, and a platform.

In some implementations, the platform device to support breasts is configured to support a user's breast, such as while the user is sitting or standing, to relieve pressure on the user's upper body caused by the user's breast (e.g., by the weight or mass of the user's breast).

In some implementations, the platform device to support breasts is configured to position on a user's lap during usage.

In some implementations, the platform device to support breasts is configured to position on a floor or similar surface during usage.

In some implementations, the platform device to support breasts is configured to position on a sitting surface, such as on a chair, couch, bed, floor, etc., under a user's thighs during usage.

In some implementations, the platform device to support breasts is configured to position adjacent to and/or against a user's body to provide fit and comfort during usage.

In some implementations, the platform device to support breasts is configured to adjust in length or height.

In some implementations, the platform device to support breasts is configured to transform between a usage configuration and a storage and/or carry configuration.

In some implementations, a method of using the platform device to support breasts comprises positioning the base of the platform device to support breasts on a user's lap, on a sitting surface under a user's thighs, or on another suitable surface, further positioning the platform device to support breasts adjacent to and/or against the user's body, and resting the user's breast on the platform of the platform device to support breasts.

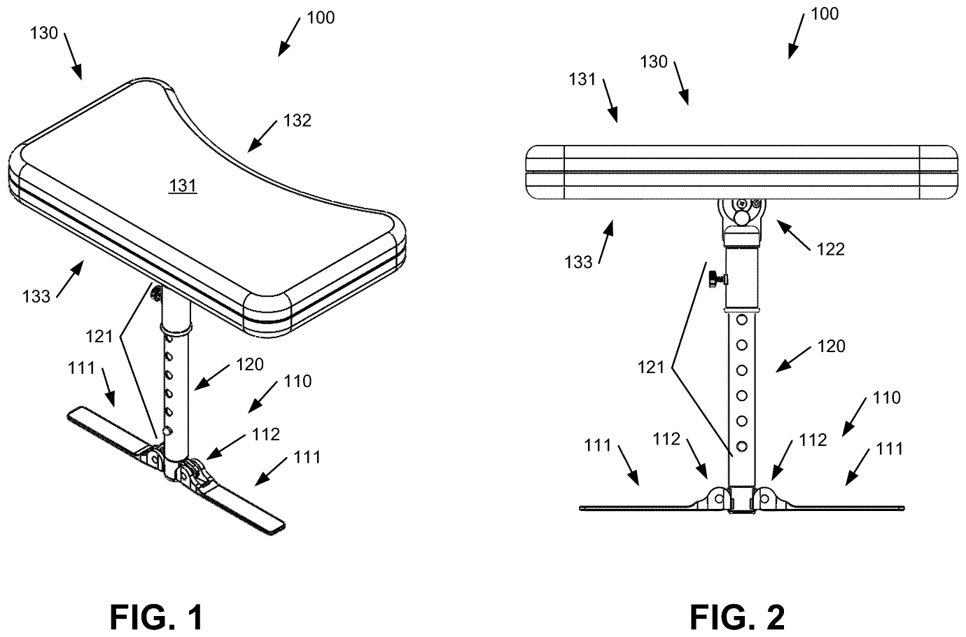

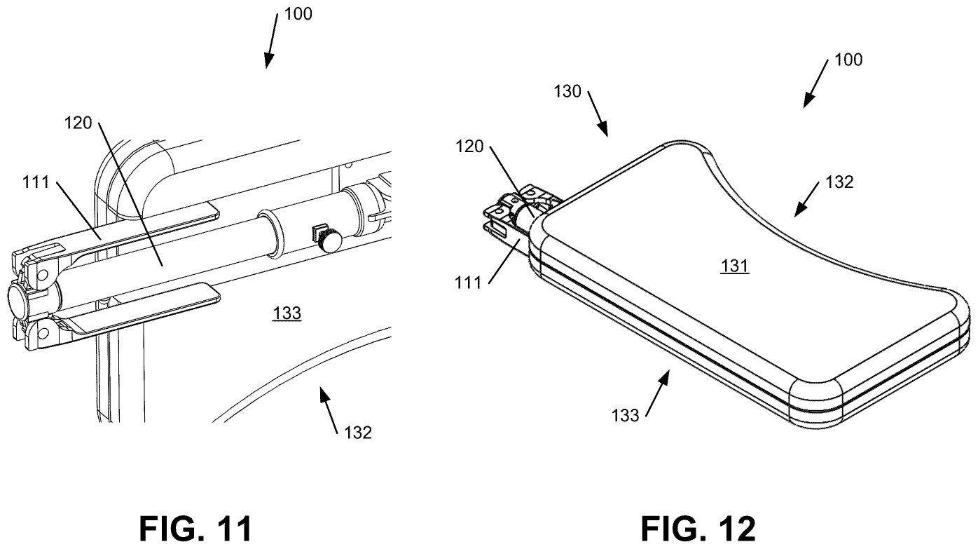

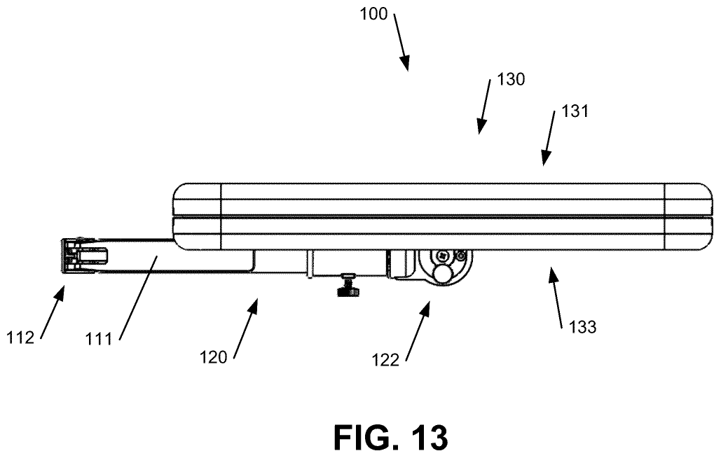

FIGS. 1-13 illustrate implementations of an example platform device to support breasts 100 according to the present disclosure. As shown in FIGS. 1 and 2, in some implementations, the platform device to support breasts 100 comprises a base 110, a vertical pole 120, and a platform 130.

In some implementations, the vertical pole 120 may be any suitable size and/or shape. For example, as shown in FIGS. 1 and 2, in some implementations, the vertical pole 120 is generally tubular or cylindrical. In some implementations, the vertical pole 120 is elongated.

As shown in FIGS. 1 and 2, in some implementations, the vertical pole 120 extends between the base 110 and the platform 130. In some implementations, the vertical pole 120 extends generally vertically when the platform device to support breasts 100 is positioned for use with the base 110 at the bottom and/or downward and the platform 130 at the top and/or upward. In some implementations, the vertical pole 120 may extend in any other suitable direction.

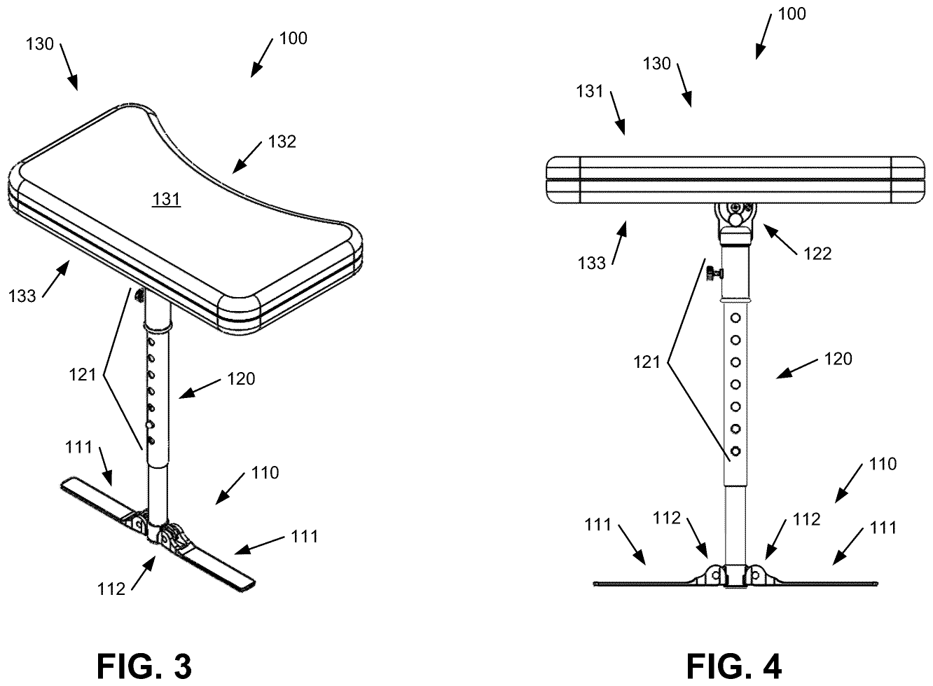

In some implementations, the vertical pole 120 is adjustable in length, such as by an adjustment mechanism 121 (e.g., a detent mechanism) as shown in FIGS. 1 and 3. In some implementations, the vertical pole 120 can thereby adjust between a shorter length or height, such as shown in FIGS. 1 and 2, and a longer length or height, such as shown in FIGS. 3 and 4.

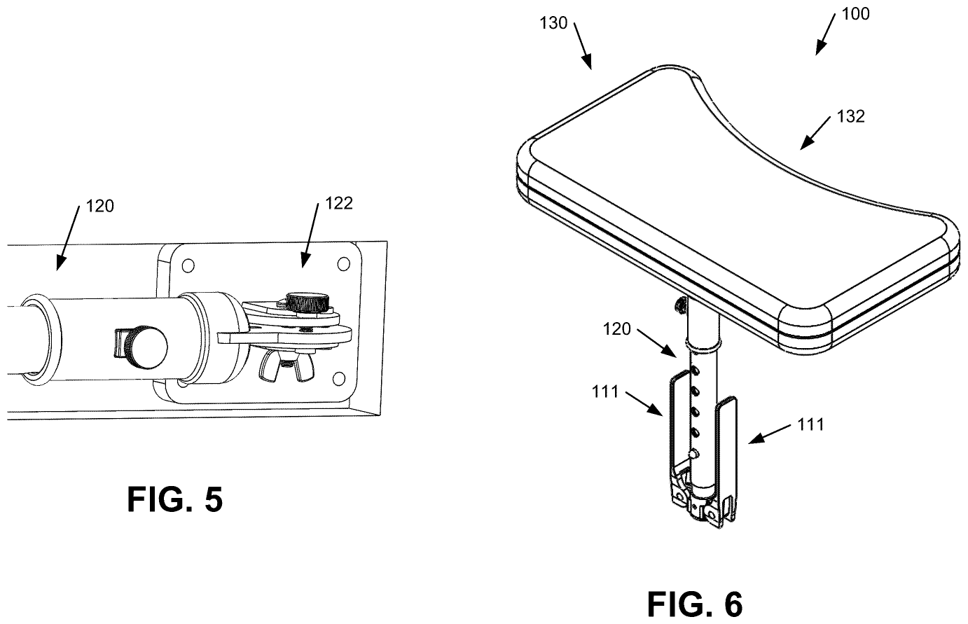

In some implementations, the vertical pole 120 is moveably and/or rotatably attached to the platform 130, such as by an attachment mechanism 122 as shown in FIG. 5. In some implementations, the vertical pole 120 can thereby fold or reposition from a generally vertical usage position, such as shown in FIGS. 1-4, to a generally horizontal storage and/or carry position, such as shown in FIGS. 10-13.

In some implementations, the base 110 may be any suitable size and/or shape. For example, as shown in FIGS. 1 and 2, in some implementations, the base 110 is generally rectangular. In some implementations, the base 110 is also generally flat.

As shown in FIGS. 1 and 2, in some implementations, the base 110 comprise one or more feet 111. In some implementations, the feet 111 may be any suitable size and/or shape. For example, as shown in FIGS. 1 and 2, in some implementations, the feet 111 are generally rectangular. In some implementations, the feet 111 are also generally flat.

In some implementations, the feet 111 are each moveably and/or rotatably attached to the vertical pole 120, such as by an attachment mechanism 112 as shown in FIGS. 1 and 2. In some implementations, the feet 111 can thereby fold or reposition from a generally horizontal usage position extending from the vertical pole 120, such as shown in FIGS. 1-4, to a generally vertical storage and/or carry position extending from and adjacent to the vertical pole 120, such as shown in FIGS. 6-13.

In some implementations, the base 110 (i.e., including the feet 111) is also moveably and/or rotatably attached to the vertical pole 120. In some implementations, the base 110 can thereby rotate or reposition from a usage position, wherein the feet 111 can extend unfolded parallel to the elongated length of the platform 130, such as shown in FIGS. 1-4, 6, and 7, to a storage and/or carry position, wherein the feet 111 can position folded and adjacent/against the bottom surface 133 of the platform 130, such as shown in FIGS. 8-13.

In some implementations, the base 110 is configured to sit on a floor or similar surface and support the platform device to support breasts 100 in a usage position. In some implementations, the base 110 is configured to sit on a user's lap and support the platform device to support breasts 100 in a usage position.

In some implementations, the base 110 is configured to sit on a sitting surface, such as on a chair, couch, bed, floor, etc., under a user's thighs and support the platform device to support breasts 100 in a usage position. For example, in some implementations, the base 110 is configured such that the user's thighs may support, hold, stabilize, etc. the base 110 (e.g., by the feet 111) on the sitting surface.

In some implementations, the base 110 may be configured to sit on any other suitable surface and support the platform device to support breasts 100 in a usage position.

In some implementations, the platform 130 may be any suitable size and/or shape. For example, as shown in FIGS. 1 and 2, in some implementations, the platform 130 is generally rectangular or rectangular prism shaped.

In some implementations, the platform 130 comprises a generally flat, horizontal surface 131 (e.g., a top or upper surface). In some implementations, the surface 131 is configured (e.g., sized and/or shaped) for a user's breasts to rest upon during usage of the platform device to support breasts 100.

In some implementations, the platform 130, such as the surface 131, may be cushioned. For example, in some implementations, the platform 130 may be cushioned with a memory foam or other suitable cushioning.

As shown in FIGS. 1 and 2, in some implementations, the platform 130 further comprises at least one side (“curved side”) 132 having a curved shape, such as a U-shape or similar shape. In some implementations, the curved side 132 is generally crescent shaped or semi-circular (or arc) shaped.

In some implementations, the curved side 132 defines a cutout (or cutout portion) in the platform 130 and/or surface 131.

In some implementations, the curved side 132 may be configured to position adjacent to and/or against a user's body, such as an area of the body below the user's breasts while the user's breast rest upon the platform 130. For example, the curved side 132 may be configured to provide fit, comfort, etc. during usage of the platform device to support breasts 100.

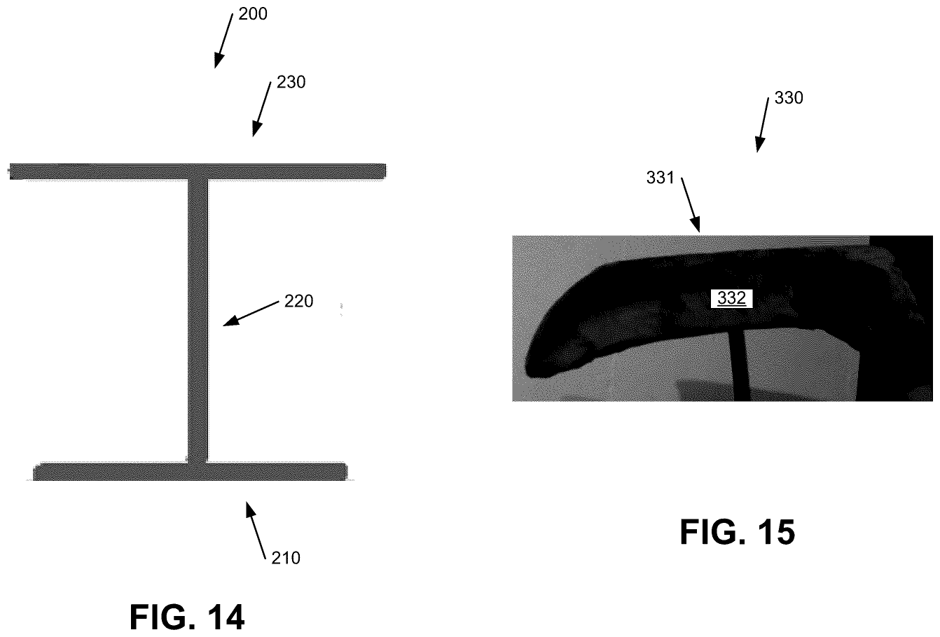

FIG. 14 illustrates another implementation of an example platform device to support breasts 200 according to the present disclosure.

In some implementations, the platform device to support breasts 200 is generally the same or similar to the platform device to support breasts 100 described herein for FIGS. 1-13. For example, as shown in FIG. 14, in some implementations, the platform device to support breasts 200 comprises a base 210, a vertical pole 220, and a platform 230 that are generally the same or similar respectively to the base 110, the vertical pole 120, and the platform 130 of the platform device to support breasts 100 of FIGS. 1-13.

FIG. 15 illustrates another implementation of an example platform 330 of the platform device to support breasts 100, 200 according to the present disclosure.

In some implementations, the platform 330 is generally the same or similar to the platform 130, 230 described herein for FIGS. 1-13 and 14 respectively. For example, as shown in FIG. 15, in some implementations, the platform 330 comprises a surface 331 and a curved side 332 that are generally the same or similar respectively to the surface 131 and the curved side 132 of the platform device to support breasts 100 of FIGS. 1-13.

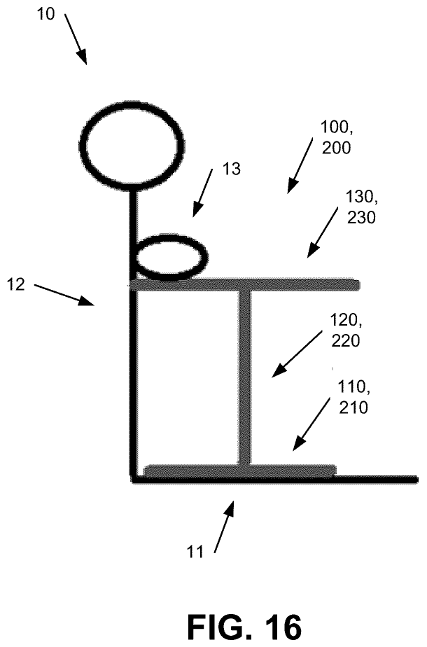

In some implementations, the platform device to support breasts 100, 200 is configured to support a user's breast, such as while the user is sitting or standing, to relieve pressure on the user's upper body caused by the user's breast (e.g., by the weight or mass of the user's breast), such as shown in FIG. 16.

In some implementations, the platform device to support breasts 100, 200 is configured to position on a user's lap during usage.

In some implementations, the platform device to support breasts 100, 200 is configured to position on a floor or similar surface during usage.

In some implementations, the platform device to support breasts 100, 200 is configured to position on a sitting surface, such as on a chair, couch, bed, floor, etc., under a user's thighs during usage. For example, in some implementations, the platform device to support breasts 100, 200 is configured such that the user's thighs may support, hold, stabilize, etc. the platform device to support breasts 100, 200 (e.g., by the base 110) on the sitting surface.

In some implementations, the platform device to support breasts 100, 200 is configured to position adjacent to and/or against a user's body to provide fit, comfort, etc. during usage.

In some implementations, the platform device to support breasts 100, 200 is configured to adjust in length or height.

In some implementations, the platform device to support breasts 100, 200 is configured to transform between a usage configuration and a storage and/or carry configuration.

In some implementations, the platform device to support breasts 100, 200 comprises any suitable dimensions. For example, in some implementations, as discussed above and further below, the platform device to support breasts 100, 200 is configured (e.g. sized, as well as shaped) to support a user's breast, such as while the user is sitting (or standing) with the base 110 positioned on the user's lap (or on a sitting surface under a user's thighs or on a floor or other suitable surface) such as shown in FIG. 16.

In some implementations, the platform device to support breasts 100, 200 is composed of any suitable materials. For example, in some implementations, the base 110 and/or vertical pole 120 the platform device to support breasts 100, 200 may be composed of a metal or plastic material. In some implementations, the platform 130 may be composed of a plastic and/or foam material.

In some implementations, the platform device to support breasts 100, 200 can have any suitable appearance, such as the example appearances shown in the figures.

In some implementations, an example method of using the platform device to support breasts 100, 200, with respect to the above-described figures, comprises positioning the base 110, 210 of the platform device to support breasts 100, 200 on a user's 10 lap 11, on a floor or similar surface, on a sitting surface (such as on a chair, couch, bed, floor, etc.) under a user's thighs 11, or on any other suitable surface.

In some implementations, the method comprises further positioning the platform device to support breasts 100, 200 adjacent to and/or against the user's 10 upper body (or upper torso) 12 such that the platform 130, 230 is positioned adjacent to and/or against the user's 10 upper body 12 under (or below) the user's 10 breast 13.

In some implementations, the platform 130 is positioned such that the curved side 132 positions adjacent to and/or against the user's 10 upper body 12 and the top surface 131 positions below the user's 10 breast 13. In some implementations, the height of the vertical pole 120, 220 is adjusted (e.g., lengthened or shortened) accordingly to position the platform 130, 230 under the user's 10 breast 13 or at an otherwise desired height and/or position.

In some implementations, the method comprises resting the user's 10 breast 13 on the platform 130, 230 such that the user's 10 breast 13 are supported by the platform device to support breasts 100, 200, such as shown in FIG. 16. In some implementations, pressure on the user's 10 upper body 12 caused by the user's 10 breast 13 is thereby relieved.

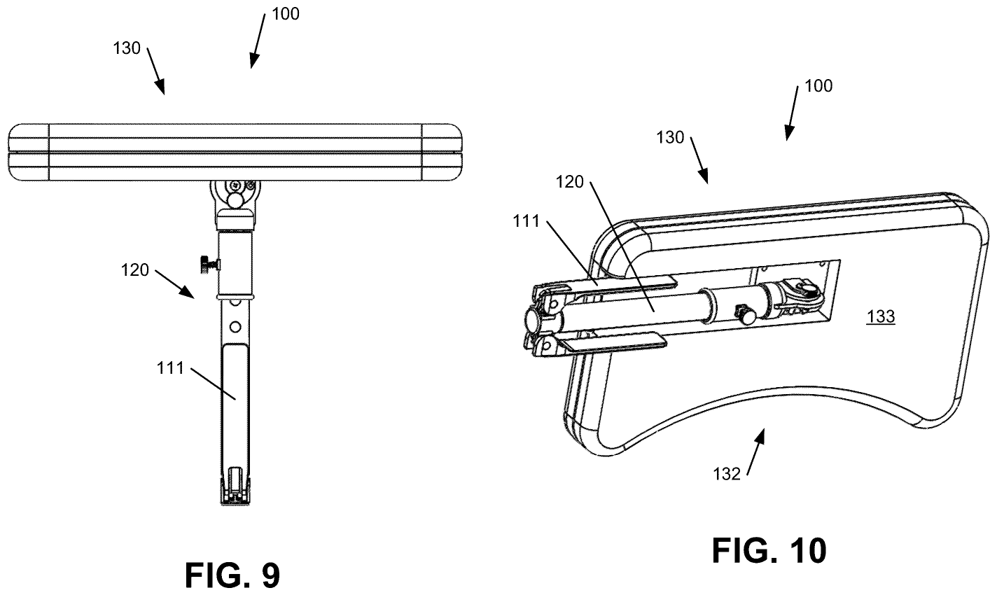

In some implementations, the method may further comprise placing the platform device to support breasts 100, 200 in a usage configuration before use. For example, in some implementations, the vertical pole 120 is rotated, unfolded, or otherwise repositioned from a horizontal position adjacent to and/or against the bottom surface 133 of the platform 130, such as shown in FIGS. 10-13, to a vertical position extending from the platform 130, such as shown in FIGS. 8 and 9.

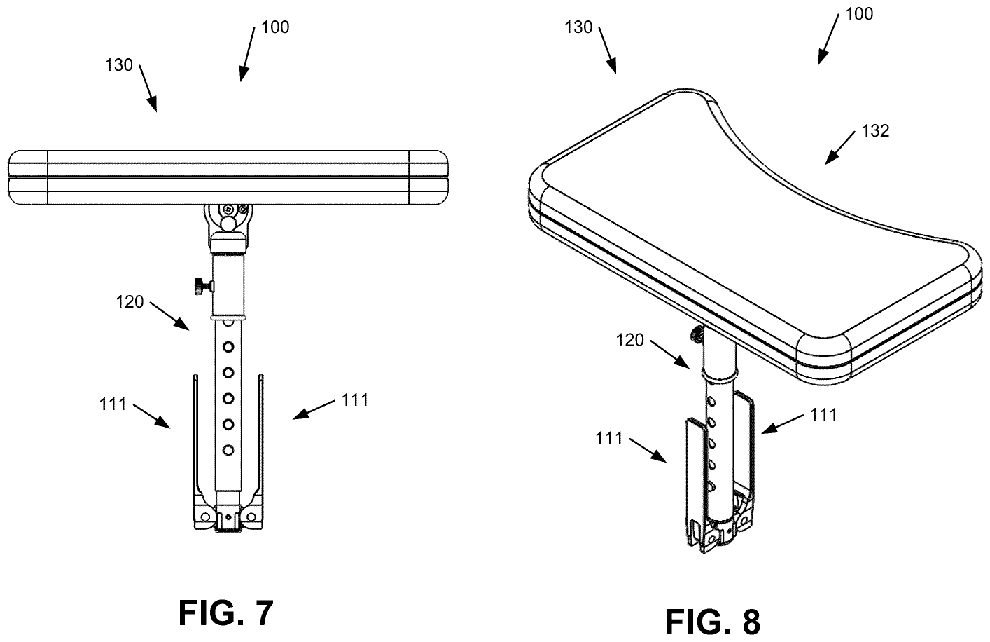

In some implementations, the base 110 is rotated about the vertical pole 120 from the storage and/or carry position, such as shown in FIGS. 8 and 9, to the usage position, such as shown in FIGS. 6 and 7.

In some implementations, the feet 111 are rotated, unfolded, or otherwise repositioned from the vertical storage and/or carry position, such as shown in FIGS. 6 and 7, to the horizontal usage position, such as shown in FIGS. 1-4.

In some implementations, the method may further comprise placing the platform device to support breasts 100, 200 in a storage and/or carry configuration after use. For example, in some implementations, the platform device to support breasts 100 is placed in the storage and/or carry configuration by reversing the foregoing steps of placing the platform device to support breasts 100 in the usage configuration.

The figures, including photographs and drawings, comprised herewith may represent one or more implementations of the platform device to support breasts.

Details shown in the figures, such as dimensions, descriptions, etc., are exemplary, and there may be implementations of other suitable details according to the present disclosure.

Reference throughout this specification to “an embodiment” or “implementation” or words of similar import means that a particular described feature, structure, or characteristic is comprised in at least one embodiment of the present invention. Thus, the phrase “in some implementations” or a phrase of similar import in various places throughout this specification does not necessarily refer to the same embodiment.

Many modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

While operations may be depicted in the drawings in a particular order, this should not be understood as requiring that such operations be performed in the particular order shown or in sequential order, or that all illustrated operations be performed, to achieve desirable results.

1. A platform device to support breasts comprising:
a base, a vertical pole, and a platform wherein:
the vertical pole extends between the base and the platform;
the base is sized and shaped to sit on a surface; and
the platform comprises a horizontal surface upon which a user's breasts can rest and at least one side of the platform comprises a curved shape forming a cutout portion of the platform that can position adjacent to the user's upper body.

2. The platform device to support breasts of claim 1 wherein the vertical pole is adjustable in length.

3. The platform device to support breasts of claim 1 wherein the base is sized and shaped to sit on a floor.

4. The platform device to support breasts of claim 1 wherein the base is sized and shaped to sit on a user's lap.

5. The platform device to support breasts of claim 1 wherein the base is sized and shaped to sit under a user's thighs.

6. The platform device to support breasts of claim 1 wherein the horizontal surface of the platform is flat.

7. The platform device to support breasts of claim 1 wherein the platform comprises cushioning.

8. The platform device to support breasts of claim 1 wherein the base comprises a pair of feet extending from the vertical pole.

9. The platform device to support breasts of claim 8 wherein the feet are moveably attached to the vertical pole such that the feet can be moved to a horizontal extended position and a vertical folded position.

10. The platform device to support breasts of claim 8 wherein the feet are moveably attached to the vertical pole such that the feet can be rotated around the pole.

11. The platform device to support breasts of claim 1 wherein the vertical pole is moveably attached to the platform such that the vertical pole can be moved to a vertical extended position and a horizontal folded position.

12. A method of using the platform device to support breasts of claim 1 comprising:
placing the base on a surface;
positioning the cutout portion of the platform adjacent to a user's upper body; and
resting the user's breasts on the horizontal surface of the platform.

13. The method of claim 12 wherein placing the base on a surface comprises placing the base on a floor.

14. The method of claim 12 wherein placing the base on a surface comprises placing the base on the user's lap.

15. The method of claim 12 wherein placing the base on a surface comprises placing the base under the user's thighs.

16. A platform device to support breasts comprising:
a base, a vertical pole, and a platform wherein:
the vertical pole extends between the base and the platform and is adjustable in length;
the vertical pole is moveably attached to the platform such that the vertical pole can be moved to a vertical extended position and a horizontal folded position;
the base comprises a pair of feet moveably attached to the vertical pole such that the feet can be moved to a horizontal extended position and a vertical folded position and can be rotated around the pole;
the base is sized and shaped to sit on a surface and under a user's thighs; and
the platform comprises a horizontal surface upon which a user's breasts can rest and at least one side of the platform comprises a curved shape forming a cutout portion of the platform that can position adjacent to the user's upper body.

17. A method of using the platform device to support breasts of claim 16 comprising:
placing the base on a surface and under a user's thighs;
positioning the cutout portion of the platform adjacent to the user's upper body; and
resting the user's breasts on the horizontal surface of the platform.

18. The method of claim 17 further comprising adjusting the length of the vertical pole to place the platform at a desired height.

19. A method of using the platform device to support breasts of claim 16 comprising:
moving the vertical pole to a vertical extended position; and
moving the feet of the base to a horizontal extended position.

20. A method of using the platform device to support breasts of claim 16 comprising:
moving the feet of the base to a vertical folded position; and
moving the vertical pole to a horizontal folded position.
